SEQUENCE LISTING (3 sequences)

SEQ ID NO: 1 - protein, 190 aa, Homo sapiens
MDHSHHMGMSYMDSNSTMQPSHHHPTTSASHSHGGGDSSMMMMPMTFYFGFKNVELLFSGLVINTAGEMAGAFVAVFLLAMFYEGLKIARESLLRKSQVSIRYNSMPVPGPNGTILMETHKTVGQQMLSFPHLLQTVLHIIQVVISYFLMLIFMTYNGYLCIAVAAGAGTGYFLFSWKKAVVVDITEHCH

SEQ ID NO: 2 - protein, 190 aa, Homo sapiens
MDHSHHMGMSYMDSNSTMQPSHHHATTSASHSHGGGDSSMMMMPMTFYFGFKNVELLFSGLVINTAGEMAGAFVAVFLLAMFYEGLKIARESLLRKSQVSIRYNSMPVPGPNGTILMETHKTVGQQMLSFPHLLQTVLHIIQVVISYFLMLIFMTYNGYLCIAVAAGAGTGYFLFSWKKAVVVDITEHCH

SEQ ID NO: 3 - DNA, 51 bp, Homo sapiens
  misc_feature: s is c or g
taccatgcaaccttctcaccatcacscaaccacttcagcctcacactccca

(19) United States
(12) Patent Application Publication
(10) Pub. No.: US 2017/0037471 A1
(43) Pub. Date: Feb. 9, 2017

(54) METHODS FOR DIAGNOSING & TREATING COPPER-DEPENDENT DISEASES

(71) Applicant: Duke University, Durham, NC (US)

(72) Inventors: Dennis J. Thiele, Chapel Hill, NC (US); Byung-Eun Kim, Bethesda, MD (US)

(21) Appl. No.: 15/106,873

(22) PCT Filed: Dec. 23, 2014
(86) PCT No.: PCT/US14/72093

§ 371 Date: Jun. 21, 2016

Related U.S. Application Data
(60) Provisional application No. 61/920,266, filed on Dec. 23, 2013.

(51) Int. Cl. C12Q 1/68; A61K 33/34

(52) U.S. Cl. CPC C12Q 1/6883; A61K 33/34; C12Q 2600/156

(57) ABSTRACT

Described are methods and materials for diagnosing a subject's predisposition for cardiovascular disease by detecting a copper deficiency genetic marker, as well as methods of alleviating Cu transport impairment. Specifically, the Cu deficiency genetic marker may be within the gene encoding a transmembrane Cu transporter protein (Ctri) or its regulatory sequences.

### CROSS REFERENCE TO RELATED APPLICATIONS

This application claims priority to U.S. Provisional Application No. 61/920,066, filed Dec. 23, 2013, which is incorporated herein by reference in their entirety.

### STATEMENT REGARDING FEDERALLY SPONSORED RESEARCH

This invention was made with government support under RO1 DK074192 awarded by the National Institutes of Health. The United States government has certain rights in the invention.

### TECHNICAL FIELD

The present disclosure relates to the field of diagnosing and treating copper-dependent diseases using a copper deficiency genetic marker, as well as methods of alleviating Cu transport impairment.

### BACKGROUND

Copper (Cu) is a trace element important for proper growth, development and health in humans. It serves as a biochemical co-factor that is essential for enzymes and proteins that function in energy production, connective tissue formation, blood vessel maturation, oxidative stress protection, peptide hormone processing and other important physiological roles. Cu is particularly important in the heart, where there is an abundance of cytochrome oxidase and superoxide dismutase, which are two Cu-dependent enzymes that are necessary to drive the energy production and oxidative stress protection that is imperative for normal cardiac function. Furthermore, it has been well established that dietary Cu deficiency in animals can lead to cardiomyopathy and heart dysfunction.

Early assessment of disorders, such as Cu-dependent diseases, may present the best opportunity to improve disease prognosis and intervention. With the development of genetic testing, it is possible to identify genetic markers that will be indicative of a predisposition to develop disease or indicative of a disease state. Specifically, there is a need to identify genetic markers that are predictive of Cu-dependent diseases, given its role in a multitude of critical biological functions, including cardiac function.

### SUMMARY

Provided herein is a method for determining a subject's predisposition for a Cu-dependent disease. The method may comprise providing a nucleic acid-containing sample obtained from a subject. A determination may be made as to whether the sample comprises a Cu-dependent marker. A Cu-dependent marker associated with a Cu-dependent disease may be rs2233915, wherein the presence of the marker indicates that the subject has a predisposition for a Cu-dependent disease. The Cu-dependent marker may be within the gene encoding Ctr1 or its regulatory sequences.

The marker of predisposition to a Cu-dependent disease may be amplified. The marker may be detected by amplifying nucleic acids. The marker may be detected by sequencing. The marker may be amplified using primers. The amplified nucleic acid of the marker may be detected by hybridizing an oligonucleotide probe to the amplified product. The oligonucleotide probe may incorporate a detectable label. The oligonucleotide may comprise the single nucleotide polymorphism (SNP) rs2233915.

A marker may be common in one geographical, ethnic, gender, and/or age group, and may be more rare, or non-existent, in another. The marker may indicate a person of African descent has a predisposition to a Cu-dependent disease. Furthermore, the marker may indicate a person of West African or Nigerian decent has a predisposition to a Cu-dependent disease.

Also provided herein is a method that may indicate a subject's predisposition to cardiovascular disease. Cardiovascular disease may be associated with cardiac hypertrophy or cardiomyopathy. Furthermore, the method may indicate a subject's predisposition to a disease mediated by abnormal enzyme activity, wherein Cu is a cofactor for the enzyme. Specifically, the method may indicate a subject's predisposition to a disease mediated by abnormal cytochrome oxidase activity, superoxide dismutase activity, or the combination thereof.

In another aspect, the disclosure provides a method of treating a Cu-dependent disease, comprising administering to the subject an effective amount of Cu, such as a Cu dietary supplement. Another method of treatment may comprise administering to the subject a cysteine protease inhibitor, such as a Cathepsin L inhibitor, in amounts effective to treat the Cu-dependent disease. The Cathepsin L inhibitor may be Z-FY(tBu)-DMK or E64d. Furthermore, the method of treatment may include the combination of both Cu and a cysteine protease inhibitor.

In another aspect, the disclosure provides a method of treating a subject's predisposition for a Cu-dependent disease. The method may comprise providing a nucleic acid-containing sample obtained from a subject, a determination of whether the sample comprises a Cu-dependent marker, rs2233915, wherein the presence of the marker indicates that the subject has a predisposition for a Cu-dependent disease. The method further comprises administering to the subject an effective amount Cu, cysteine protease inhibitor, or combination thereof.

### BRIEF DESCRIPTION OF THE DRAWINGS

FIG. 1 depicts a model for copper homeostasis in mammalian cells. The Ctr1 high affinity Cu transporter at the plasma membrane is shown transporting Cu⁺ (blue ball) into the cytoplasm. Intracellular Cu is bound by protein chaperones (Atox1 and CCS) for delivery to the secretory compartment via handoff to Cu pumps (ATP7A or ATP7B) or to superoxide dismutase (SOD1). ATP7A and ATP7B can also export excess Cu by delivery across the plasma membrane or to intracellular vesicles which fuse with the plasma membrane. Excess Cu in the cytoplasm is bound by metallothioneins (MTs). On a genetic level, the MTF1 transcription factor regulates expression of some of the nuclear genes in response to elevated Cu levels. Mitochondrial cytochrome oxidase (CCO) is a Cu-dependent enzyme that generates energy through oxidative phosphorylation and Sco1/2, Cox17 and Cox11 are involved in the delivery of Cu to CCO.

FIG. 2 illustrates the overall structure of human Ctr1 and the position of the Ctr1P25A polymorphism. Ctr1 has three membrane-spanning domains, a short carboxy-terminus in the cytoplasm and an amino-terminal extracellular domain, denoted the ectodomain. Methionine and histidine rich regions in the extracellular domain are depicted in the lighter gray areas. Branched structures indicate the presence of two glycosylated amino acids residues. The Ctr1 variant (Ctr1P25A) expressed from the rs2233915 SNP is diagrammatically shown with the Proline to Alanine substitution at amino acid 25.

FIG. 3 demonstrates that the Ctr1P25A variant leads to increased presence of a truncated form of Ctr1, which results in impaired Cu transport in mouse embryonic fibroblasts (MEFs).

FIG. 4 displays the effects of Ctr2 on the cleavage of Ctr1 and subsequent accumulation of Cu.

FIG. 5 establishes that B cells isolated from Yoruba individuals expressing the Ctr1p25A variant have Ctr1 cleavage and Cu accumulation phenotypes similar to MEFs that express the Ctr1P25A variant.

FIG. 6 indicates a high frequency of Ctr1P25A variant SNP allele in cardiac patients that are African American.

FIG. 7 represents the effect of Cathepsin L on cleavage of the Ctr1 ectodomain.

FIG. 8 demonstrates the protection of the Ctr1P25A ectodomain with a Cathepsin L inhibitor.

FIGS. 9A-9D demonstrate that changing the functional status of Ctr1 precipitates cardiac dysfunction. FIG. 9A shows pregnant mice that are heterozygous for the Ctr1 gene in the heart die after ˜5 rounds of pregnancy, whereas wild type mice survive multiple rounds of pregnancy. FIG. 9B shows mice that are heterozygous for Ctr1 in the heart have defects associated with cardiomyopathy, including decreased fractional shortening and decreased heart rate. FIG. 9C shows that tissue sectioning of mice heterozygous for Ctr1 in heart tissue shows enlargement of cardiomyocytes compared to wild type mice, a phenotype of dilated cardiomyopathy. FIG. 9D shows that mice that were engineered to harbor a systemic heterozygosity for Ctr1 (Ctr1+/− in all tissues) are more prone to cardiac hypertrophy when reared on copper deficient food than wild type mice.

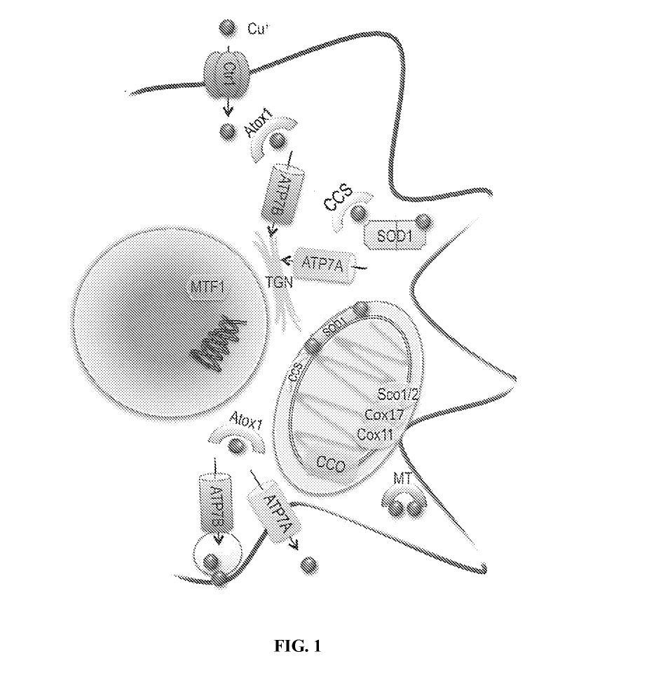

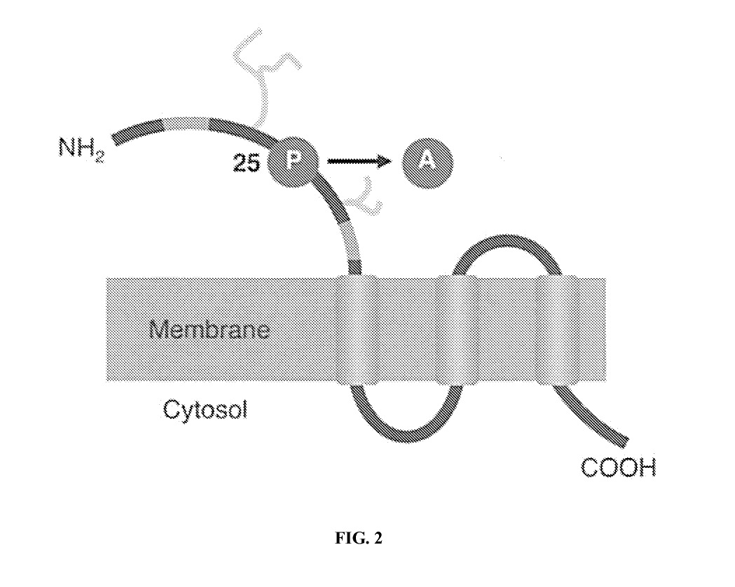

### DETAILED DESCRIPTION

The inventors have made the discovery that there is an association between Cu-dependent diseases and a genetic marker. This genetic marker resides in the gene encoding for the protein Ctr1 (“Ctr1”), which is a transmembrane Cu transporter protein. The marker, which is a single nucleotide polymorphism (SNP), resulting in a proline to alanine substitution, leads to an impaired variant of Ctr1. The ecto-domain of this variant has increased sensitivity to cysteine proteases. This increased protease sensitivity may lead to decreased Cu import capacity. The identification of this Cu-dependent marker in a subject may be useful in predicting a person's predisposition in developing a disease mediated by abnormal Cu levels within a cell. In addition, knowledge of this particular marker may allow one to customize the prevention or treatment in accordance with the subject's genetic profile. Early detection of a Cu-dependent disease marker will allow the subject to take preventive measures that would abrogate manifestation and progression of Cu-mediated diseases (e.g. cardiovascular disease).

The ability to target populations expected to show the highest clinical benefit, based on genetic profile, may enable the repositioning of already marketed drugs, the rescue of drug candidates whose clinical development has been discontinued as a result of safety or efficacy limitations, which may be patient sub-group-specific, and/or an accelerated and less costly development of candidate therapeutics.

The methods and materials described below use genetic analysis to determine the presence of a Cu-dependent disease marker and reveal whether a subject may be predisposed to a disease mediated by impaired Cu transport.

### 1. Definitions

“Administration” or “administering,” as used herein, refers to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

“Cathepsin L” as used herein, refers to a lysosomal cysteine proteinase that plays a role in intracellular protein catabolism. This proteinase may also be referred to as “Cathepsin L1”. As used herein the term Cathepsin L encompasses any ortholog, variant, or functional fragment thereof. Multiple alternatively spliced transcript variants have been found for the gene CTSL1 which encodes the Cathepsin L1 protein; these include the sequences described in NCBI Reference Sequence Nos. NM_001912.1, NM_001912.2, NM_001912.3 and NM_001912.4. The Cathepsin L protein may include, for example, the sequence described in NCBI Reference Sequence Nos. NP_001903.1 and NP_666023.1.

“Co-administered,” as used herein, refers to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks or one month of each other. Suitably, compounds or agents are co-administered during the period in which each of the compounds or agents are exerting at least some physiological effect and/or has remaining efficacy.

“Ctr1”, as used herein, refers to a membrane associated, homotrimeric protein that transports reduced copper (Cu(I)) into cells. As used herein, the term Ctr1 encompasses any ortholog, variant, or functional fragment thereof. Ctr1 can include, for example, the sequence described in NCBI Reference Sequence No. NP_001850.

“Ctr2”, as used herein, refers to a membrane associated, oligomeric protein that plays a role in regulating copper uptake into cells along with Ctr1. As used herein, the term Ctr2 encompasses any ortholog, variant, or functional fragment thereof. Ctr2 can include, for example, the sequence described in NCBI Reference Sequence No. NP_001851.1.

“Cu-dependent disease”, as used herein, refers to any disease where abnormal intracellular Cu levels are involved in disease manifestation and/or progression. Examples of a Cu-dependent disease may include, but are not limited to, diseases of the cardiovascular system, diseases of the immune system, diseases mediated by abnormal angiogenesis, diseases mediated by abnormal cellular respiration, diseases mediated by abnormal oxidative stress. Examples of cardiovascular diseases may include, but are not limited to, cardiac hypertrophy and cardiomyopathy. Additionally, examples may include, but are not limited to, diseases mediated by abnormal enzyme activity, wherein Cu is a cofactor for the enzyme. Representative enzymes may include, but are not limited to, superoxide dismutase, cytochrome oxidase, amino acid oxidase, ceruloplasmin, catechol oxidase, dopamine-β-monooxygenase, protein-lysine 6-oxidase, peptidylglycine monooxygenase, and metallothionein.

“Effective amount,” as used herein, refers to a dosage of compounds or compositions effective for eliciting a desired effect. This term as used herein may also refer to an amount effective at bringing about a desired in vivo effect in an animal, mammal, or human, such as reducing symptoms of Cu-dependent diseases.

“Fragment” as used herein, may mean a portion of a reference peptide or polypeptide or nucleic acid sequence.

“Label” or “detectable label” as used herein, may mean a moiety capable of generating a signal that allows the direct or indirect quantitative or relative measurement of a molecule to which it is attached. The label may be a solid such as a microtiter plate, particle, microparticle, microscope slide; an enzyme; an enzyme substrate; an enzyme inhibitor; coenzyme; enzyme precursor; apoenyzme; fluorescent substrate; pigment; chemiluminescent compound; luminescent substance; coloring substance; magnetic substance; or a metal particle such as a gold colloid; a radioactive substance such as 125I, 131I, 32P, 3H, 35S, or 14C; a phosphorylated phenol derivative such as nitrophenyl, luciferin derivative, or dioxetane derivative; or the like. The enzyme may be a dehydrogenase; an oxidoreductase such as reductase or oxidase; a transferase that catalyzes the transfer of functional groups, such as an amino; carboxy, methyl, acyl, or phosphate group, a hydrolase that may hydrolyze a bond such as an ester, glycoside, ether, or peptide bond; a lyases; an isomerase; or a ligase. The enzyme may also be conjugated to another enzyme.

The enzyme may be detected by enzymatic cycling. For example, when the detectable label is an alkaline phosphatase, a measurement may be made by observing the fluorescence or luminescence generated from a suitable substrate, such as an umbelliferone derivative. The umbelliferone derivative may comprise 4-methyl-umbellipheryl phosphate.

The fluorescent or chemiluminescent label may be a fluorescein isothiocyanate, a rhodamine derivative such as rhodamine β isothiocyanate or tetramethyl rhodamine isothiocyanate; a dancyl chloride (5-(dimethylamino)-1-naphtalenesulfonyl chloride); a dancyl fluoride; a fluorescamine (4′-phenylspiro[2-benzofuran-3,2′-furan]-1,3′-dione); a phycobiliprotein such as a phycocyanine or physoerythrin; an acridinium salt; a luminol compound such as lumiferin, luciferase, or aequorin; imidazoles; an oxalic acid ester; a chelate compound of rare earth elements such as europium (Eu), terbium (Tb) or samarium (Sm); or a coumarin derivative such as 7-amino-4-methylcoumarin.

The label may also be a hapten, such as adamantine, fluoroscein isothiocyanate, or carbazole. The hapten may allow the formation of an aggregate when contacted with a multi-valent antibody or (strep)avidin containing moiety. The hapten may also allow easy attachment of a molecule to which it is attached to a solid substrate.

The label may be detected by quantifying the level of a molecule attached to a detectable label, such as by use of electrodes; spectrophotometric measurement of color, light, or absorbance; or visual inspection.

“Pharmaceutically acceptable,” as used herein, pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

“Subject,” as used herein, is intended to include human and non-human animals. In embodiments, the subject is a human. Exemplary human subjects include a human that is a descendent of Africa. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

“Treat” or “treating,” as used herein, refers to a subject having a disorder refers to administering a regimen to the subject, e.g., the administration of a cysteine inhibitor-based therapeutic and/or another agent, such that at least one symptom of the disorder is healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

### 2. Method of Diagnosis

Provided herein is a method of determining a subject's predisposition for a Cu-dependent disease. This predisposition may be associated with a genetic marker. The detection of the marker in a nucleic acid-containing sample from the subject may be indicative of a predisposition for a disease that manifests from a Cu deficiency.

a. Sample

The sample may be any sample that comprises nucleic acid from a subject. The sample may be any cell type, tissue or bodily fluid. The sample may be nucleic acid isolated from a cell, tissue and/or bodily fluid. The nucleic acid may be DNA or RNA. The nucleic acid may be genomic. The sample may be used directly as obtained from the subject or following pretreatment to modify a character of the sample. Pretreatment may include extraction, concentration, inactivation of interfering components, and/or the addition of reagents.

The cell types, tissues, and fluid may include sections of tissues such as biopsy and autopsy samples, frozen sections taken for histologic purposes, blood, plasma, serum, sputum, stool, tears, mucus, saliva, hair, and skin. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose. Archival tissues, such as those having treatment or outcome history, may also be used. Nucleic acid purification may not be necessary.

b. Cu-Dependent Disease Marker

The Cu-dependent disease marker may be a genetic marker. The marker may be a deletion, substitution, insertion, or a polymorphism. The polymorphism may be a SNP. The SNP may be rs2233915. The marker may be within the Ctr1 gene. The SNP may be a polymorphism that results in a proline to alanine substitution at position 25 of the Ctr1 protein (of the SLC31A1 open reading frame). rs2233915 may be a missense mutation whereby a cytosine is replaced with a guanine. The rs2233915 (C/G) may be in the following sequence:

(SEQ ID NO: 3)
TACCATGCAACCTTCTCACCATCAC[C/G]CAACCACTTCAGCCTCA
CACTCCCA.

Within a population, the marker may be assigned a minor allele frequency. There may be variations between subject populations. A marker that is common in one geographical or ethnic group may be rarer in another. The marker may be overrepresented or underrepresented in a group of subjects. Subjects may be divided into groups on the basis of age, sex/gender, and/or race.

i. SLC31A1 Polymorphisms

The methods comprise providing a nucleic acid-containing sample obtained from the subject; and detecting a Ctr1 nucleotide sequence encoding a Ctr1 protein, or fragment thereof, such as SEQ ID NO:1 or SEQ ID NO:2, or a fragment thereof. See Table 1. The presence of SEQ ID NO: 1 indicates that the subject does not have a predisposition to Cu-dependent disease. The presence of SEQ ID NO: 2 indicates that the subject does have a predisposition to a Cu-dependent disease and could be a candidate for a treatment tailored for the subject.

TABLE 1

| Protein | Sequence | SEQ ID |
|---|---|---|
| NP_001851.1, | MDHSHHMGMSYMDSNSTMQ | SEQ ID NO: 1 |
| high affinity | PSHHHPTTSASHSHGGGDS | |
| copper uptake | SMMMMPMTFYFGFKNVELL | |
| protein 1 | FSGLVINTAGEMAGAFVAV | |
| [Homo sapiens] | FLLAMFYEGLKIARESLLR | |
| | KSQVSIRYNSMPVPGPNGT | |
| | ILMETHKTVGQQMLSFPHL | |
| | LQTVLHIIQVVISYFLMLI | |
| | FMTYNGYLCIAVAAGAGTG | |
| | YFLFSWKKAVVVDITEHCH | |
| p.Pro25Ala | MDHSHHMGMSYMDSNSTMQ | SEQ ID NO: 2 |
| high affinity | PSHHHATTSASHSHGGGDS | |
| copper uptake | SMMMMPMTFYFGFKNVELL | |
| protein 1 | FSGLVINTAGEMAGAFVAV | |
| [Homo sapiens] | FLLAMFYEGLKIARESLLR | |
| SNP | KSQVSIRYNSMPVPGPNGT | |
| | ILMETHKTVGQQMLSFPHL | |
| | LQTVLHIIQVVISYFLMLI | |
| | FMTYNGYLCIAVAAGAGTG | |
| | YFLFSWKKAVVVDITEHCH | |

SEQ ID NO:2 is represented in the Yoruban tribe in Nigeria, and is also represented in DNA samples from African American patients in the Duke CATHGEN database and sample collection. Expression of a gene including this SNP in mouse embryonic fibroblasts produces a Ctr1 protein that is present almost exclusively in a form in which the ecto-domain has been cleaved. See Examples.

c. Detection

The Cu-dependent disease marker may be detected in the sample. Many methods are available for detecting a marker in a subject and may be used in conjunction with the herein described methods. These methods include large-scale SNP genotyping, exonuclease-resistant nucleotide detection, solution-based methods, genetic bit analyses, primer guided nucleotide incorporation, allele specific hybridization, and other techniques. Any method of detecting a marker may use a labeled oligonucleotide.

i. Large Scale SNP Genotyping

Large scale SNP genotyping may include any of dynamic allele-specific hybridization (DASH), microplate array diagonal gel electrophoresis (MADGE), pyrosequencing, oligonucleotide-specific ligation, or various DNA “chip” technologies such as Affymetrix SNP chips. These methods may require amplification of the target genetic region. Amplification may be accomplished via polymerase chain reaction (PCR).

ii. Exonuclease-Resistant Nucleotide

Cu-dependent disease markers may be detected using a specialized exonuclease-resistant nucleotide, as described in U.S. Pat. No. 4,656,127, which is incorporated herein by reference. A primer complementary to the allelic sequence immediately 3′ to the polymorphic site may be permitted to hybridize to a target molecule obtained from the subject. If the polymorphic site on the target molecule contains a nucleotide that is complementary to the particular exonuclease-resistant nucleotide derivative present, then that derivative may be incorporated onto the end of the hybridized primer. Such incorporation may render the primer resistant to exonuclease, and thereby permit its detection. Since the identity of the exonuclease-resistant derivative of the sample may be known, a finding that the primer has become resistant to exonuclease reveals that the nucleotide is present in the polymorphic site of the target molecule was complementary to that of the nucleotide derivative used in the reaction. This method may not require the determination of large amounts of extraneous sequence data.

iii. Solution-Based Method

A solution-based method may be used to determine the identity of a Cu-dependent disease marker, as described in PCT Application No. WO91/02087, which is herein incorporated by reference. A primer may be employed that is complementary to allelic sequences immediately 3′ to a polymorphic site. The method may determine the identity of the nucleotide of that site using labeled dideoxynucleotide derivatives that, if complementary to the nucleotide of the polymorphic site, will become incorporated onto the terminus of the primer.

iv. Genetic Bit Analysis

Genetic bit analysis may use mixtures of labeled terminators and a primer that is complementary to the sequence 3′ to a polymorphic site. A labeled terminator may be incorporated, wherein it is determined by and complementary to, the nucleotide present in the polymorphic site of the target molecule being evaluated. The primer or the target molecule may be immobilized to a solid phase.

v. Primer-Guided Nucleotide Incorporation

A primer-guided nucleotide incorporation procedure may be used to assay for a Cu-dependent disease marker in a nucleic acid, as described in Nyren, P. et al., Anal. Biochem. 208:171-175 (1993), which is herein incorporated by reference. Such a procedure may rely on the incorporation of labeled deoxynucleotides to discriminate between bases at a polymorphic site. In such a format, the signal is proportional to the number of deoxynucleotides incorporated, thus polymorphisms that occur in runs of the same nucleotide may result in signals that are proportional to the length of the run.

vi. Allele Specific Hybridization

Allele specific hybridization may be used to detect a Cu-dependent disease marker. This method may use a probe capable of hybridizing to a target allele. The probe may be labeled. A probe may be an oligonucleotide. The target allele may have between 3 and 50 nucleotides around the marker. The target allele may have between 5 and 50, between 10 and 40, between 15 and 40, or between 20 and 30 nucleotides around the marker. A probe may be attached to a solid phase support, e.g., a chip. Oligonucleotides may be bound to a solid support by a variety of processes, including lithography. A chip may comprise more than one allelic variant of a target region of a nucleic acid, e.g., allelic variants of two or more polymorphic regions of a gene.

vii. Other Techniques

Examples of other techniques for detecting alleles include selective oligonucleotide hybridization, selective amplification, or selective primer extension. Oligonucleotide primers may be prepared in which the known mutation or nucleotide difference is placed centrally and then hybridized to target DNA under conditions which permit hybridization if a perfect match is found. Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Allele specific amplification technology that depends on selective PCR amplification may be used in conjunction with the instant invention. Oligonucleotides used as primers for specific amplification may carry the mutation or polymorphic region of interest in the center of the molecule. Amplification may then depend on differential hybridization, as described in Gibbs et al. (1989) Nucleic Acids Res. 17:2437-2448), which is herein incorporated by reference, or at the extreme 3′ end of one primer where, under appropriate conditions, mismatch can prevent, or reduce polymerase extension.

Direct DNA sequencing, either manual sequencing or automated fluorescent sequencing may detect sequence variation. Another approach is the single-stranded conformation polymorphism assay (SSCP), as described in Orita M, et al. (1989) Proc. Natl. Acad. Sci. USA 86:2766-2770, which is incorporated herein by reference. The fragments that have shifted mobility on SSCP gels may be sequenced to determine the exact nature of the DNA sequence variation. Other approaches based on the detection of mismatches between the two complementary DNA strands include clamped denaturing gel electrophoresis (CDGE), as described in Sheffield V C, et al. (1991) Am. J. Hum. Genet. 49:699-706, which is incorporated herein by reference; heteroduplex analysis (HA), as described in White M B, et al. (1992) Genomics 12:301-306, which is incorporated herein by reference; and chemical mismatch cleavage (CMC) as described in Grompe M, et al., (1989) Proc. Natl. Acad. Sci. USA 86:5855-5892, which is herein incorporated by reference. A review of currently available methods of detecting DNA sequence variation can be found in a review by Grompe (1993), which is incorporated herein by reference. Grompe M (1993) Nature Genetics 5:111-117. Once a mutation is known, an allele specific detection approach such as allele specific oligonucleotide (ASO) hybridization can be utilized to rapidly screen large numbers of other samples for that same mutation. Such a technique can utilize probes that may be labeled with gold nanoparticles to yield a visual color result as described in Elghanian R, et al. (1997) Science 277:1078-1081, which is herein incorporated by reference.

A rapid preliminary analysis to detect polymorphisms in DNA sequences can be performed by looking at a series of Southern blots of DNA cut with one or more restriction enzymes, preferably with a large number of restriction enzymes.

d. Amplification

Any method of detection may incorporate a step of amplifying the Cu-dependent disease marker. A Cu-dependent disease marker may be amplified and then detected. Nucleic acid amplification techniques may include cloning, PCR, allele specific PCR (ASA), ligase chain reaction (LCR), nested polymerase chain reaction, self-sustained sequence replication, transcriptional amplification system, and Q-Beta Replicase, as described in Kwoh, D. Y. et al., 1988, Bio/Technology 6:1197, which is incorporated herein by reference.

Amplification products may be assayed by size analysis, restriction digestion followed by size analysis, detecting specific tagged oligonucleotide primers in reaction products, allele-specific oligonucleotide (ASO) hybridization, allele specific 5′ exonuclease detection, sequencing, and/or hybridization.

Nucleic acid primers and/or oligonucleotides may be used in conjunction with any of the herein described methods and/or kits. For example, the oligonucleotide may be synthesized and selected to hybridize to an amplified product. The oligonucleotide may comprise a detectable label.

PCR-based detection methods may include amplification of a single marker or a plurality of markers simultaneously. For example, PCR primers may be synthesized and selected to generate PCR products that do not overlap in size and may be analyzed simultaneously. Alternatively, one may amplify different markers with primers that are differentially labeled. Each marker may then be differentially detected. Hybridization-based detection methods may allow the differential detection of multiple PCR products in a sample.

Subjects identified as having the genetic marker that predisposes them for having a Cu-dependent disease may be candidates to receive treatment for the Cu-dependent disease. Treatment may be preventative or therapeutic for the Cu-dependent disease.

### 3. Method of Treatment

Provided herein is a method of treating a subject having the Cu-dependent disease marker. The subject may be predisposed to a Cu-dependent disease, or the subject may have a Cu-dependent disease. The subject may be undergoing treatment for cardiovascular disease, cancer, etc. The method of treatment may be the administration of an effective amount of Cu to a subject in need thereof. The method of treatment may be the administration of an effective amount of cysteine protease inhibitor to a subject in need thereof. The method of treatment may be the administration of an effective amount of Cu and cysteine protease inhibitor to a subject in need thereof. Additionally, the method of treatment may be providing a nucleic acid containing sample from the subject, determining whether Cu-dependent disease marker is present within the sample and administering an effective amount of Cu and/or cysteine protease inhibitor if the marker is present in the subject.

The treatment of a subject with a particular therapeutic may be monitored by determining protein, mRNA, and/or transcriptional level of a gene. The gene may be for the protein Ctr1. Depending on the level detected, the therapeutic regimen may be maintained or adjusted. The effectiveness of treating a subject with an agent may comprise (1) obtaining a pre-administration sample from a subject prior to administration of the agent; (2) detecting the level, amount or size of a protein, RNA or DNA in the pre-administration sample; (3) obtaining one or more post-administration samples from the subject; (4) detecting the level of expression, size or activity of the protein, RNA or DNA in the post-administration sample; (5) comparing the level of expression or activity of the protein, RNA or DNA in the pre-administration sample with the corresponding protein, RNA or DNA in the post-administration sample, respectively; and (6) altering the administration of the agent to the subject accordingly.

Cells of a subject may be obtained before and after administration of a therapeutic to detect the level of expression of genes other than the gene of interest to verify that the therapeutic does not increase or decrease the expression of genes that could be deleterious. Verification may be accomplished by transcriptional profiling. mRNA from cells exposed in vivo to a therapeutic and mRNA from the same type of cells that were not exposed to the therapeutic may be reverse transcribed and hybridized to a chip containing DNA from many genes. The expression of genes in the treated cells may be compared against cells not treated with the therapeutic.

Appropriate therapy may be essential steps in the management of a Cu-dependent disease. Therapeutics for any given subject in any given setting may be based on periodic isolation and identification of disease indices.

a. Cu Supplements

The methods may comprise treating a subject with an effective amount of Cu. Cu may be in the form of a supplement. The Cu supplement may be formulated using any pharmaceutically acceptable form of the mineral, including its salts. It may be formulated into capsules, tablets, powders, gels or liquids. The Cu supplement may be formulated as powders, for example, for mixing with consumable liquids such as milk, juice, water or consumable gels or syrups for mixing into other dietary liquids or foods.

The Cu supplement formulation may include tablets, capsules, granules and bulk powders. Tablets may contain suitable binders, lubricants, diluents, disintegrating agents, coloring agents, flavoring agents, flow-inducing agents and melting agents. Liquid oral dosage forms include aqueous solutions, emulsions, suspensions, solutions and/or suspensions reconstituted from non-effervescent granules and effervescent preparations reconstituted from effervescent granules. Such liquid oral dosage forms may contain, for example, suitable solvents, preservatives, emulsifying agents, suspending agents, diluents, sweeteners, melting agents, and coloring and flavoring agents.

The Cu supplement may be used for daily administration. It may be formulated for once-daily administration, but may be formulated in multiple portions or as time release compositions for more or less frequent administration; for example, the Cu supplement may be formulated as two tablets for twice daily administration, or as a sustained release capsule for administration every three days.

b. Cysteine Protease Inhibitors

In some embodiments, the methods comprise treating a subject with an effective amount of cysteine protease inhibitor, such as an inhibitor of Cathepsin L, B, C, F, H, K, V, O, S or W. Exemplary cysteine protease inhibitors include Cathepsin L inhibitors. A Cathepsin L inhibitor may be any compound capable of reducing or eliminating the activity of Cathepsin L, such as a small molecule or an antibody. The Cathepsin L inhibitor may further comprise a small interfering RNA (siRNA) capable of interfering with the expression of Cathepsin L.

Certain small molecule inhibitors of Cathepsin L are known. These include, for example, the following:

Z-FF-FMK, also known as Cbz-Phe-Phe-fluoromethylketone or “Cathepsin L Inhibitor I”, having the chemical name benzyl (1-((4-fluoro-3-oxo-1-phenylbutan-2-yl)amino)-1-oxo-3-phenylpropan-2-yl)carbamate, CAS No. 108005-94-3;

Z-FY-CHO, also known as Cbz-Phe-Tyr-CHO or “Cathepsin L Inhibitor II”, having the chemical name benzyl (1-((1-(4-hydroxyphenyl)-3-oxopropan-2-yl)amino)-1-oxo-3-phenylpropan-2-yl)carbamate, CAS No. 167498-29-5;

Z-FY(tBu)-DMK, also known as Cbz-Phe-Tyr(tBu)-diazomethylketone or “Cathepsin L Inhibitor III”, having the chemical name benzyl (1-((1-(4-(tert-butoxy)phenyl)-4-diazo-3-oxobutan-2-yl)amino)-1-oxo-3-phenylpropan-2-yl)carbamate;

E-64, also known as trans-Epoxysuccinyl-L-leucylamido(4-guanidino)butane, L-trans-3-Carboxyoxiran-2-carbonyl-L-leucylagmatine, or N-(trans-Epoxysuccinyl)-L-leucine 4-guanidinobutylamide, CAS No. 66701-25-5;

E-64C, also known as (2S,3S)-trans-Epoxysuccinyl-L-leucylamido-3-methylbutane, CAS No. 76684-89-4; and

E-64D, also known as (2S,3S)-trans-Epoxysuccinyl-L-leucylamido-3-methylbutane ethyl ester, CAS No. 88321-09-9.

Other Cathepsin L inhibitors are known, as disclosed, for example, at http://www.scbt.com/chemicals-table-cathepsin_1_inhibitors.html.

c. Formulations

While compounds such as Cu and cysteine protease inhibitors may be administered alone in the various methods described herein, they may also be presented singly or together in one or more pharmaceutical compositions (e.g., formulations). In each composition the compounds may be formulated with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Accordingly, the methods described herein include administration of one or more pharmaceutical compositions, as discussed herein, in which a compound such as Cu or a cysteine protease inhibitor is admixed together with one or more pharmaceutically acceptable carriers, excipients, buffers, adjuvants, stabilizers, or other materials, as described herein. Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods known in the art of pharmacy. Such methods include the step of bringing into association the active compound(s) with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and nonaqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate and nanoparticulate systems which are designed to target the active compound to blood components or one or more organs.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases. Further formulations suitable for inhalation include those presented as a nebulizer.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

d. Dosages

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

A suitable dosage range for the cysteine protease inhibitor may be between 0.01 mg per kg of body weight per day (mg/kg/day) to about 100 mg/kg/day, between 0.5 mg/kg/day and 90 mg/kg/day, between 1 mg/kg/day and 80 mg/kg/day, between 5 mg/kg/day and 75 mg/kg/day, between 10 mg/kg/day and 70 mg/kg/day, between 15 mg/kg/day and 65 mg/kg/day, between 20 mg/kg/day and 60 mg/kg/day, between 25 mg/kg/day and 55 mg/kg/day, between 30 mg/kg/day and 50 mg/kg/day, between 35 mg/kg/day and 50 mg/kg/day, or between 40 mg/kg/day and 45 mg/kg/day.

### 4. Kit

Provided herein is a kit, which may be used for diagnosing, monitoring or treating a Cu-dependent disease. The kit may comprise a nucleic acid sample collecting method. The kit may also comprise a means for determining a marker in the Ctr1 gene sequence, a nucleic acid for use as a positive control, and/or nucleic acid sampling means. The nucleic sampling means may include substrates, such as filter paper, nucleic acid purification reagents, such as reaction buffer, polymerase, and dNTPs. Marker detection means may also be included in the kit. Such means may include, specific restriction enzymes, marker specific oligonucleotides, and degenerate oligonucleotide primers for PCR. The positive control may be used for sequence comparison.

The kit may also comprise one or more containers, such as vials or bottles, with each container containing a separate reagent. The kit may further comprise written instructions, which may describe how to perform or interpret an assay or method described herein.

### EXAMPLES

### Example 1

### Ctr1^{P25A} Mutation Analysis in MEF Cells

Mouse embryonic fibroblasts (MEFs) were transfected with nothing (Ctr1), and empty vector (Vec), the wild type Ctr1 complementary DNA (WT) or the Ctr1^{P25A} complementary DNA (P25A). Wild type MEFs (Ctr1+/+) were used as a control for the abundance of the full length and truncated forms of Ctr1.

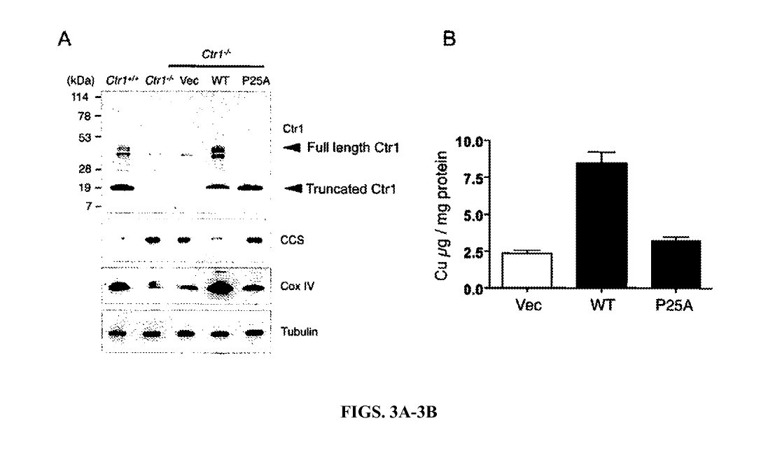

FIG. 3A depicts immunoblot analysis of protein extracts from control and transfected MEFs. Full length and truncated Ctr1 are indicated with arrows and the sizes of molecular weight markers (in kiloDaltons) on the left side of the image. Blots containing SDS-PAGE fractionated protein extracts were also probed with antibodies against CCS, CoxIV and Tubulin. Tubulin served as a protein loading control. CCS protein levels are known to be elevated in Cu-deficient cells relative to Cu replete cells and they are elevated in the Ctr1P25A expressing and Vector transfected cells relative to cells expressing wild type Ctr1. CoxIV levels are known to be elevated in Cu replete cells and lower in Cu-limited. Taken together, the increased levels of truncated Ctr1, increased levels of CCS and decreased levels of CoxIV in CtrP25A expressing cells, as compared to wild type Ctr1 expressing cells, demonstrates decreased Cu transport activity associated with truncated Ctr1.

FIG. 3B exhibits Ctr1−/− MEFs stably transfected with wild type Ctr1 expression plasmid, vector, or a Ctr1P25A expression plasmid analyzed for steady-state Cu levels by Inductively Coupled Plasmon Resonance—Mass Spectrometry (ICP-MS). Cu levels are represented as micrograms of Cu per mg of protein extract. WT Ctr1 expressing cells accumulate significantly more Cu than Ctr1^{P25A} expressing cells.

### Example 2

### Ctr1 Ectodomain Cleavage in Ctr2^{+/+}, Ctr2^{+/-} and Ctr2^{-/-} MEFs

MEFs from wild type, Ctr2^{+/-}, and Ctr2^{-/-} littermates were cultured in medium supplemented with 10% fetal bovine serum and harvested at 90% confluency. Total proteins were isolated from the cells by homogenizing cells in ice cold PBS supplemented with 1% Triton-X, 0.1% SDS, and 1 mM EDTA. Cell debris was removed by centrifugation and the total amounts of soluble proteins were quantified in each sample. Equal amounts of proteins were separated on a tris/glycine gradient gel, transferred to nitrocellulose membrane and blocked with 5% non-fat milk in Tris buffered saline supplemented with 0.05% Tween (TBST) for 1 h. Membranes were incubated with anti-Ctr1 antibody (1:1000) followed by anti-rabbit-HRP coupled antibody (1:5000) and bands detected by enhanced chemiluminescent substrate. Anti-Tubulin antibody was used as loading control.

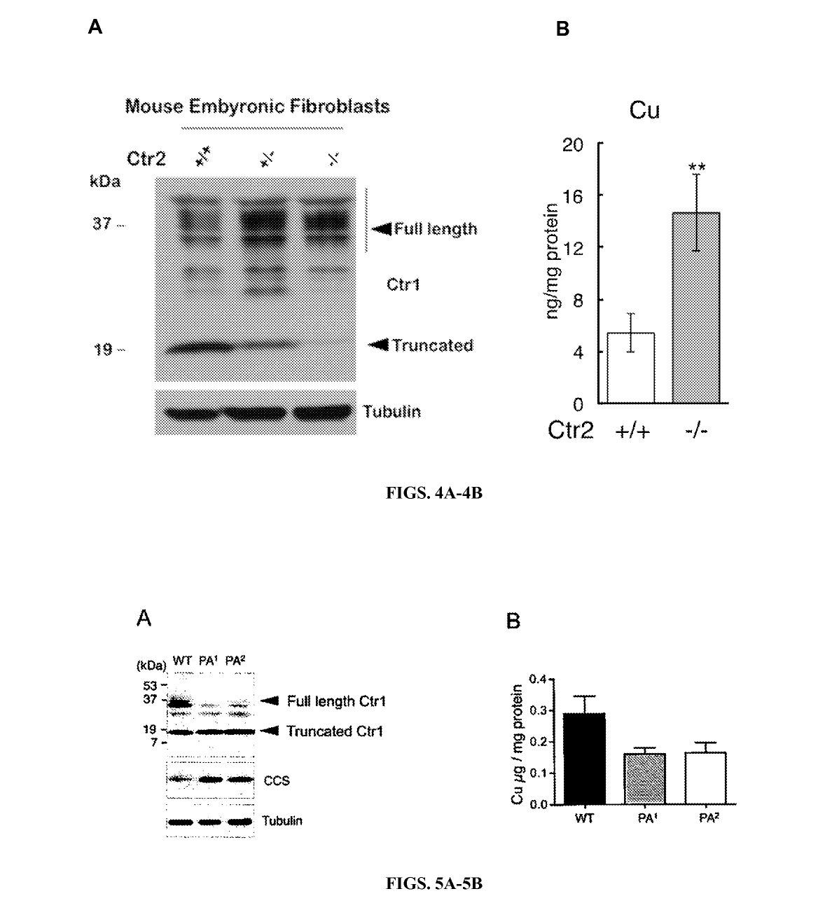

FIG. 4A illustrates immunoblotting of MEFs from Wild type (Ctr2+/+), heterozygous (Ctr2+/−) and knock out cells (Ctr2−/−) with anti-Ctr1 antibody and anti-Tubulin antibody as a control. Loss of Ctr2 results in a gene-dosage-dependent decrease in Ctr1 cleavage, as indicated by the abundance of the truncated form of Ctr1 (Truncated) versus the full length form (Full length).

FIG. 4B indicates the effects of Ctr2 expression on Cu accumulation within MEFs. MEFs from wild type and Ctr2−/− littermates were cultured in medium supplemented with 10% fetal bovine serum and treated with 200 μM Cisplatin for 2 hours. Cells were rinsed three times with ice cold PBS before the cells were scraped and divided into two tubes; one for measuring metal concentration and one for protein quantification. Cell pellets from four independent cultures for each treatment groups were digested in concentrated nitric acid supplemented with 30% hydrochloric acid for 1 hour at 85° C. and mixed with ddH2O. Cu concentration in the digested samples were measured by ICP-MS and normalized to the total amount of protein in the sample. The loss of Ctr2 expression exhibited increased Cu accumulation within cells.

### Example 3

### Ctr1^{P25A} Mutation Analysis in B Cells from Yoruba Individuals

FIG. 5A illustrates immunoblot analysis of protein extracts from B cells isolated from Yoruba individuals expressing wild type Ctr1 or two different individuals expressing Ctr1^{P25A} protein (PA¹, PA²). Full length and truncated Ctr1 are indicated with arrows and the sizes of molecular weight markers (in kiloDaltons) on the left side of the image. Blots containing SDS-PAGE fractionated protein extracts were also probed with antibodies against CCS and Tubulin. Tubulin served as a protein loading control. The truncated form of Ctr1 is significantly more prevalent in both the PA1 and PA2 B cell lines as compared to the wild type control cell line. Additionally, CCS levels are elevated in both the PA1 and PA2 B cell lines, as compared to the wild type control cell line. This suggests potential Cu limitation in both PA1 and PA2 B cell lines.

FIG. 5B examines the steady-state Cu levels of the wild type, PA1 and PA2 B cell lines. ICP-MS was used to analyze Cu levels in the different B cell line samples. Cu levels were shown as micrograms of Cu per mg of cell protein extract. The data demonstrates that the WT Ctr1 expressing B cell line accumulates significantly more Cu then either of the two Ctr1P25A expressing B cell lines.

### Example 4

### Genotyping of Ctr1 rs2233915 SNP Presence in Cardiac Patients

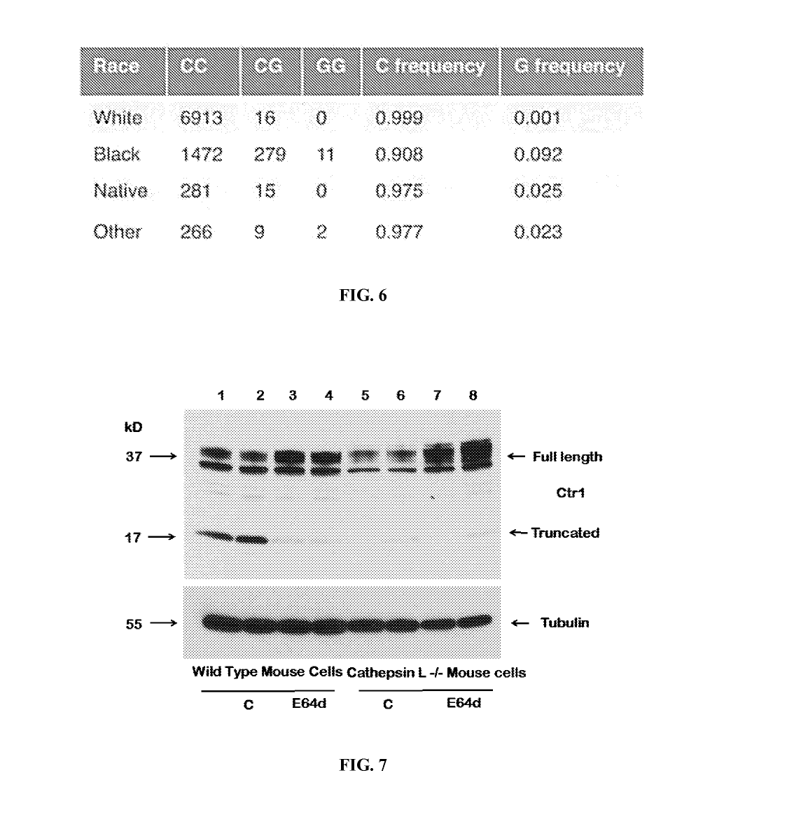

The rs2233915 SNP was genotyped from DNA samples from the Duke University CATHGEN 9500, a cohort of 9,500 individuals referred for evaluation of heart disease. The data was evaluated with respect to patient phenotypes that include sick sinus syndrome, cardiomyopathy and survival. FIG. 6 represents the frequency of the Ctr1^{P25A} allele in self-declared White, Black, Native American and Other races. While the frequency of the polymorphism was almost undetectable in the CATHGEN Caucasian population (16 heterozygotes of 6,929 DNA samples surveyed) African American samples showed 16% heterzygosity and 0.6% homozygosity for the polymorphism of the total 1,762 samples analyzed. Moreover, there was an association with death with a significance of p=0.03 in an additive model with a hazard risk of 1.3.

### Example 5

### Ctr1 Cleavage in Cathepsin L^{-/-} Cells

Mouse embryonic fibroblasts from wild type and CatL^{-/-} littermates were cultured in medium supplemented with 10% fetal bovine serum and treated with DMSO or 10 μM of the cell permeable cysteine protease inhibitor E64d and harvested 16 hours later. Total proteins were isolated from the cells by homogenizing cells in ice cold PBS supplemented with 1% Triton-X, 0.1% SDS, and 1 mM EDTA. Cell debris was removed by centrifugation and the total amounts of soluble proteins were quantified in each sample. Equal amounts of proteins were separated on tris/glycine gradient gel, transferred to nitrocellulose membrane and blocked with 5% non-fat milk in Tris buffered saline supplemented with 0.05% Tween (TBST) for 1 h. Membranes were incubated with anti-Ctr1 antibody (1:1000) followed by anti-rabbit-HRP coupled antibody (1:5000) and bands detected by enhanced chemiluminescent substrate. Anti-Tubulin antibody was used as loading control.

FIG. 7 shows immunoblotting results of the analysis of protein extracts from wild type MEFs (lanes 1-4) and Cathepsin L knock out fibroblasts (Cathepsin L−/−, lanes 5-8) with anti-Ctr1 antibody and anti-Tubulin antibody as a loading control. Loss of Cathepsin L (lanes 5-8) results in a dramatic reduction in the levels of cleaved Ctr1. Treatment of wild type cells with E64d (10 μM), a Cathepsin L inhibitor, also results in a dramatic reduction in the levels of cleaved Ctr1 in wild type cells (lanes 4 and 5 are duplicate biological experiments) but not Cathepsin L knock out cells (lanes 7 and 8 are duplicate biological experiments). Lanes 1 and 2 are duplicate samples from untreated wild type cells and lanes 5 and 6 are duplicate samples from untreated Cathepsin L knock out cells.

### Example 6

### Ctr1^{P25A} Cleavage Protection

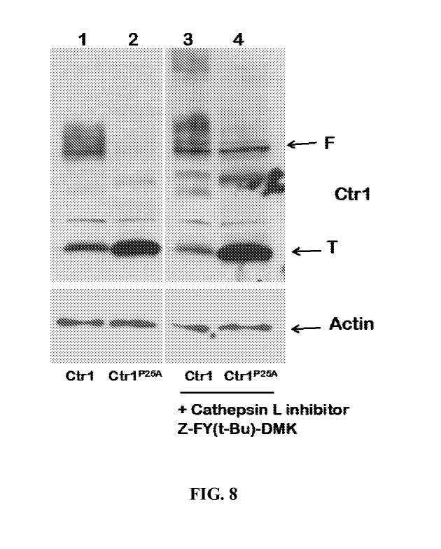

Mouse embryonic fibroblasts from Ctr1^{-/-} embryos were stably transfected with plasmids expressing either the wild type human Ctr1 protein or the human Ctr1 protein containing the proline to alanine substitution at position 25 (Ctr1^{P25A}). Cells were cultured in medium supplemented with 20% fetal bovine serum and treated with DMSO or 10 μM cysteine protease Cathepsin L, Z-FY(t-Bu)-DMK and harvested 16 hours later. Total proteins were isolated from the cells by homogenizing cells in ice cold PBS supplemented with 1% Triton-X, 0.1% SDS, and 1 mM EDTA. Cell debris was removed by centrifugation and the total amounts of soluble proteins were quantified in each sample. Equal amounts of proteins were separated on tris/glycine gradient gel, transferred to nitrocellulose membrane and blocked with 5% non-fat milk in Tris buffered saline supplemented with 0.05% Tween (TBST) for 1 h. Membranes were incubated with anti-Ctr1 antibody (1:1000) followed by anti-rabbit-HRP coupled antibody (1:5000) and bands detected by enhanced chemiluminescent substrate. Anti-Actin antibody was used as loading control. As shown in FIG. 8, cells expressing the Ctr1 proline 25 to alanine mutant protein (Ctr1^{P25A}) show a dramatic reduction in full length form that can be alleviated by treatment with Cathepsin L inhibitors.

### Example 7

### Ctr1 and Cardiac Dysfunction

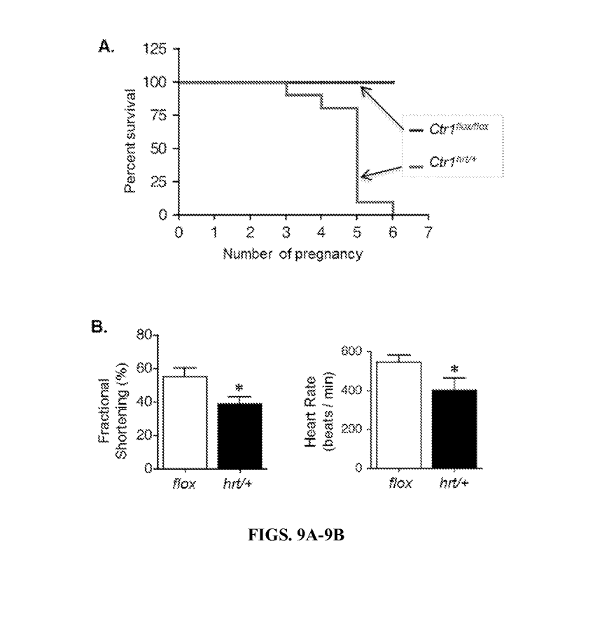

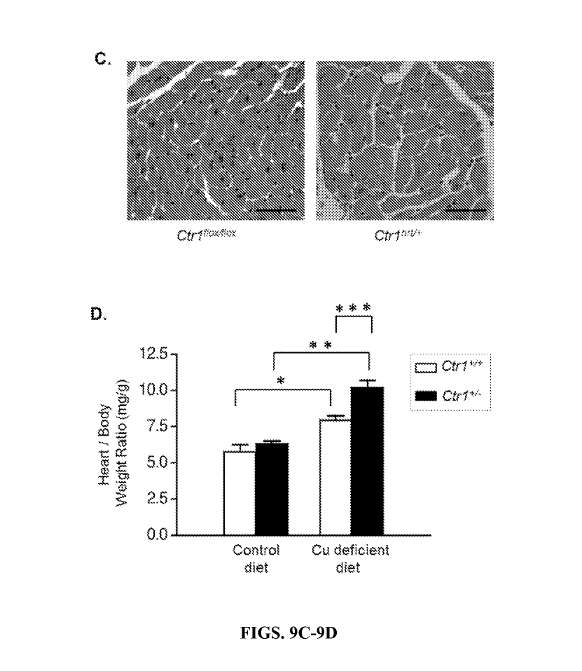

Changing the functional status of Ctr1 precipitated cardiac dysfunction. Ctr1 and Cardiac Dysfunction Pregnant female mice (˜8 months old) with either two functional copies of the Ctr1 gene (fox/fox), or with a cardiac-specific heterozygous Ctr1 state (hrt/+) (Kim et al. Cell Metabolism 11: 353-363 (2010)) were generated and mated with male mice. The mice were followed for viability over multiple rounds of pregnancy and the percent of the pregnant mice surviving birth was determined. Pregnant mice that were heterozygous for the Ctr1 gene in the heart died after about 5 rounds of pregnancy, whereas wild type mice survive multiple rounds of pregnancy. FIG. 9A.

Fractional shortening, representing left ventricular contractility, and heart rate (beats per minute) were determined by echocardiography after the fourth round of pregnancy in all mice. Mice that were heterozygous for Ctr1 in the heart had defects associated with cardiomyopathy, including decreased fractional shortening and decreased heart rate. FIG. 9B.

Thin sections of cardiac tissue were evaluated by Hematoxylin and Eosin (H and E) staining and microscopy in mice after the fourth round of pregnancy a representative field of vision photographed and shown at 40× magnification. Tissue sectioning of mice heterozygous for Ctr1 in heart tissue showed enlargement of cardiomyocytes compared to wild type mice, a phenotype of dilated cardiomyopathy. FIG. 9D.

Wild type and systemic Ctr1 heterozygous mice (Lee et al. Proc. Natl. Acad. Sci., USA 98: 6842-6847 (2001)) were reared on a normal diet or a copper deficient diet (Teklad Animal Diets, Harlan Laboratories) immediately after birth (day 1), which continued until weaning and then continued for a further two weeks after weaning. Heart weight/body weight ratio was determined by dissection of hearts and determining heart mass as compared to whole body mass. Mice that were engineered to harbor a systemic heterozygosity for Ctr1 (Ctr1^{+/-} in all tissues) were more prone to cardiac hypertrophy when reared on copper deficient food than wild type mice. FIG. 9D. Together, these data show that genetic modification of Ctr1 functional status leads to cardiomyopathy and support the observations that a SNP in the Ctr1 gene that enhances ecto-domain cleavage, and therefore diminishes copper uptake, predisposes mammals to cardiomyopathy.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A method for determining a subject's predisposition for a Cu-dependent disease, comprising: (a) providing a nucleic acid-containing sample obtained from a subject; and (b) determining whether a Cu-dependent marker is present in the sample; and wherein the marker is rs2233915, wherein the presence of the marker indicates that the subject has a predisposition for a Cu-dependent disease.

Clause 2. The method of clause 1, wherein the gene Ctr1 comprises the marker.

Clause 3. The method of clause 1, wherein the marker is detected by: (a) amplifying a nucleic acid comprising the marker; and (b) detecting the amplified nucleic acids, thereby detecting the marker.

Clause 4. The method of clause 3, wherein the marker is detected by sequencing.

Clause 5. The method of clause 3, wherein the amplified nucleic acids are detected by hybridizing an oligonucleotide probe to the amplified product.

Clause 6. The method of clause 5, wherein the probe incorporates a detectable label.

Clause 7. The method of clause 5, wherein the probe is an oligonucleotide comprising the SNP rs2233915, or fragment thereof.

Clause 8. The method of clause 1, wherein the Cu-dependent disease is a cardiovascular disease.

Clause 9. The method of clause 8, wherein the cardiovascular disease is selected from a group consisting of cardiac hypertrophy and cardiomyopathy.

Clause 10. The method of clause 1, wherein the Cu-dependent disease is mediated by abnormal enzyme activity, and wherein Cu is a cofactor for the enzyme.

Clause 11. The method of clause 1, wherein the Cu-dependent disease is mediated by abnormal cytochrome oxidase activity.

Clause 12. The method of clause 1, wherein the Cu-dependent disease is mediated by abnormal superoxide dismutase activity.

Clause 13. The method of clause 1, wherein the Cu-dependent disease is mediated by abnormal cytochrome oxidase activity and abnormal superoxide dismutase activity.

Clause 14. The method of clause 1, further comprising administering an effective amount of Cu to the subject predisposed to the Cu-dependent disease.

Clause 15. The method of clause 1, further comprising administering an effective amount of cysteine protease inhibitor to the subject predisposed to the Cu-dependent disease.

Clause 16. The method of clause 15, wherein the cysteine protease inhibitor is a Cathepsin L inhibitor.

Clause 17. The method of clause 16, wherein the Cathepsin L inhibitor is selected from the group consisting of Z-FY(tBu)-DMK and E64d.

Clause 18. A method for treating a Cu-dependent disease, comprising administering an effective amount of Cu to a subject in need thereof.

Clause 19. The method of clause 18, further comprising administering an effective amount of cysteine protease inhibitor to the subject.

Clause 20. A method for treating a Cu-dependent disease, comprising administering an effective amount of cysteine protease inhibitor to a subject in need thereof.

Clause 21. The method of clause 20, further comprising administering an effective amount of Cu to the subject.

Clause 22. A method for treating a Cu-dependent disease, comprising: (a) providing a nucleic acid-containing sample obtained from a subject; (b) determining whether a Cu-dependent marker is present in the sample, wherein the marker is rs2233915, wherein the presence of the marker indicates that the subject has a predisposition for a Cu-dependent disease; and (c) administering an effective amount of Cu if the marker is present in the subject.

Clause 23. The method of clause 22, further comprising administering an effective amount of cysteine protease inhibitor if the marker is present in the subject.

Clause 24. A method for treating a Cu-dependent disease, comprising: (a) providing a nucleic acid-containing sample obtained from a subject; (b) determining whether a Cu-dependent marker is present in the sample, wherein the marker is rs2233915, wherein the presence of the marker indicates that the subject has a predisposition for a Cu-dependent disease; and (c) administering an effective amount of cysteine protease inhibitor if the marker is present in the subject.

Clause 25. The method of clause 24, further comprising administering an effective amount of Cu if the marker is present in the subject.

Clause 26. The method of any one clause of 18, 21, 22 or 25, where the Cu is in the form of a dietary supplement.

Clause 27. A method for treating a Cu-dependent disease in an individual, comprising administering an effective amount of a cysteine protease inhibitor to an individual identified as having a Cu-dependent marker, wherein the Cu-dependent marker is rs2233915.

Clause 28. The method of clause 27, wherein the Cu-dependent disease is mediated by abnormal enzyme activity, and wherein Cu is a cofactor for the enzyme.

Clause 29. The method of clause 27, wherein the Cu-dependent disease is mediated by abnormal cytochrome oxidase activity.

Clause 30. The method of clause 27, wherein the Cu-dependent disease is mediated by abnormal superoxide dismutase activity.

Clause 31. The method of clause 27, wherein the Cu-dependent disease is mediated by abnormal cytochrome oxidase activity and abnormal superoxide dismutase activity.

Clause 32. The method of clause 27, wherein the individual is heterozygous for the Cu-dependent marker.

Clause 33. The method of clause 27, wherein the individual is homozygous for the Cu-dependent marker.

Clause 34. The method of clause 27, comprising determining that the individual is heterozygous or homozygous for the Cu-dependent marker.

Clause 35. The method of clause 27, wherein the individual suffers from cardiovascular disease.

Clause 36. The method of clause 35, wherein the cardiovascular disease is selected from a group consisting of cardiac hypertrophy and cardiomyopathy.

Clause 37. The method of clause 27, wherein the cysteine protease inhibitor is a Cathepsin L inhibitor.

Clause 38. The method of clause 37, wherein the Cathepsin L inhibitor is selected from the group consisting of Z-FY(tBu)-DMK and E64d.

Clause 39. The method of clause 27, further comprising administering an effective amount of Cu.

Clause 40. The method of clause 39, where the Cu is in the form of a dietary supplement.

Clause 41. A method for treating a Cu-dependent disease in an individual, comprising administering an effective amount of Cu to an individual identified as having a Cu-dependent marker, wherein the Cu-dependent marker is rs2233915.

Clause 42. The method of clause 41, where the Cu is in the form of a dietary supplement.

Clause 43. The method of clause 41, wherein the Cu-dependent disease is mediated by abnormal enzyme activity, and wherein Cu is a cofactor for the enzyme.

Clause 44. The method of clause 41, wherein the Cu-dependent disease is mediated by abnormal cytochrome oxidase activity.

Clause 45. The method of clause 41, wherein the Cu-dependent disease is mediated by abnormal superoxide dismutase activity.

Clause 46. The method of clause 41, wherein the Cu-dependent disease is mediated by abnormal cytochrome oxidase activity and abnormal superoxide dismutase activity.

Clause 47. The method of clause 41, wherein the individual is heterozygous for the Cu-dependent marker.

Clause 48. The method of clause 41, wherein the individual is homozygous for the Cu-dependent marker.

Clause 49. The method of clause 41, comprising determining that the individual is heterozygous or homozygous for the Cu-dependent marker.

Clause 50. The method of clause 41, wherein the individual suffers from cardiovascular disease.

Clause 51. The method of clause 50, wherein the cardiovascular disease is selected from a group consisting of cardiac hypertrophy and cardiomyopathy.

What is claimed is:

1. A method for determining a subject's predisposition for a Cu-dependent disease, comprising:
(a) providing a nucleic acid-containing sample obtained from a subject; and
(b) determining whether a Cu-dependent marker is present in the sample; and
wherein the marker is rs2233915, wherein the presence of the marker indicates that the subject has a predisposition for a Cu-dependent disease.

2. The method of claim 1, wherein the gene Ctr1 comprises the marker.

3. The method of claim 1, wherein the marker is detected by:
(a) amplifying a nucleic acid comprising the marker; and
(b) detecting the amplified nucleic acids, thereby detecting the marker.

4. The method of claim 3, wherein the marker is detected by sequencing.

5. The method of claim 3, wherein the amplified nucleic acids are detected by hybridizing an oligonucleotide probe to the amplified product.

6. The method of claim 5, wherein the probe incorporates a detectable label.

7. The method of claim 5, wherein the probe is an oligonucleotide comprising the SNP rs2233915, or fragment thereof.

8. The method of claim 1, wherein the Cu-dependent disease is a cardiovascular disease.

9. The method of claim 8, wherein the cardiovascular disease is selected from a group consisting of cardiac hypertrophy and cardiomyopathy.

10. The method of claim 1, wherein the Cu-dependent disease is mediated by abnormal enzyme activity, and wherein Cu is a cofactor for the enzyme.

11. The method of claim 1, wherein the Cu-dependent disease is mediated by abnormal cytochrome oxidase activity.

12. The method of claim 1, wherein the Cu-dependent disease is mediated by abnormal superoxide dismutase activity.

13. The method of claim 1, wherein the Cu-dependent disease is mediated by abnormal cytochrome oxidase activity and abnormal superoxide dismutase activity.

14. The method of claim 1, further comprising administering an effective amount of Cu to the subject predisposed to the Cu-dependent disease.
